(19) United States
(12) Patent Application Publication
(10) Pub. No.: US 2015/0011868 A1
(43) Pub. Date: Jan. 8, 2015

(54) MRI-Compatible Fiducial Markers and Methods for Using the Same

(71) Applicant: Adventist Health System/Sunbelt, Inc., Altamonte Springs, FL (US)

(72) Inventor: James Edmund Baumgartner, Winter Park, FL (US)

(21) Appl. No.: 14/301,529

(22) Filed: Jun. 11, 2014

Related U.S. Application Data
(60) Provisional application No. 61/833,576, filed on Jun. 11, 2013.
(60) Provisional application No. 61/949,435, filed on Mar. 7, 2014.
(60) Provisional application No. 61/949,421, filed on Mar. 7, 2014.

(51) Int. Cl. A61B 19/00; A61B 5/055

(52) U.S. Cl. CPC A61B 19/54; A61B 5/0555; A61B 2019/5454
USPC 600/414

(57) ABSTRACT

The present disclosure relates generally to an MRI-compatible fiducial marker assembly and methods of use in planning and conducting brain surgery. This invention relates to the field of anatomic orientation and positioning, particularly in relation to an implement for effectively and efficiently defining an anatomic orientation and position for conducting surgical resection of a patient's brain.

### FIELD OF THE INVENTION

The present invention relates generally to an MRI-compatible fiducial marker assembly and methods of use in planning and conducting surgical resection of the brain. This invention relates to the field of anatomic orientation and position for conducting surgical resection of a patient's brain.

### BACKGROUND OF THE INVENTION

Different medical imaging modalities provide different aspects about the condition of a brain part. In general, they contain either functional or anatomical information. Integration of multi-modal information into a single display platform is desirable in surgical planning and navigation. This is especially useful for complicated brain surgery where a morbid area is surrounded by functional areas.

Another consideration is the actual trajectory of an electrode or surgical implement relative to the critical areas. Thus, although there might be a trajectory that avoids all non-desired areas and structures, this trajectory might not be chosen if it is in close proximity to critical areas. Therefore, to avoid the risk of injuring a critical area, the implement may not be placed as close as possible to an area to be treated.

Typically, trajectories for biopsies or intra-cranial electrodes or catheters are planned prior to neurosurgery. The planning is based on target selection and on a selection of the entry point of the trajectory. To perform a minimally invasive surgical procedure, the physician plans the trajectory in consideration of critical brain areas. These areas might consist of critical and anatomical structures, such as ventricles, or physiological, vascular or functional structures.

Because the characteristic nature differs among modalities and the results are generated separately from different sources in which some of the results even require additional statistical analysis, the formats of their outputs vary. The traditional way of handling all medical image inputs is as a series of intensity fluctuation which makes integration difficult. This hinders the efficiency of clinical practice.

Additionally, due to intrinsic and unavoidable inaccuracies related to the planning and placement procedure (e.g., from image resolution registration inaccuracy, etc.), and to different levels of experience of the executing physician and/or the lack of knowledge of the patient-specific tissue configuration, as well as due to patient-specific variations in the arrangement of the tissue, for example in the case of a diseased tissue, it has been necessary to leave sufficient space between critical areas (in terms of a specific level of risk, automatically and/or manually defined) in order to ensure that an implement such as a catheter or electrode does not interfere with the critical area. These areas include, for example, eloquent brain areas, vascular structures or anatomical areas such as ventricles

Thus there exists a need for a method by which anatomic orientation and positioning, particularly in relation to an implement, for effectively and efficiently defining an anatomic orientation and position, using a single platform that can promptly display and manipulate various data that are provided by different modalities for more accurate, and therefore successful, surgical planning and navigation.

### SUMMARY OF THE INVENTION

The teachings of the present invention provide an integrated system and process that addresses the above described problems and provide a desirable solution for clinical application.

The application provides an MRI-compatible fiducial marker assembly for identifying a location in a brain comprising: a MRI-compatible base layer that is mountable on the skull of a patient's body, one or more MRI-compatible fiducial elements attached to the base layer, wherein and at least one of said MRI-compatible fiducial elements are MRI-visible fiducial markers.

The application provides an MRI-compatible fiducial marker assembly for identifying a location, the fiducial marker assembly comprising: a base layer that is mountable on a patient's skull, the base layer having opposed upper and lower primary surfaces; and at least one MRI-visible fiducial element defined by or secured to the base layer.

The application provides a method for identifying a physical location in the brain of a patient, the method comprising: providing a fiducial marker assembly including: a base layer that is mountable on a patient's skull, the base layer having opposed upper and lower primary surfaces; and at least one MRI-visible fiducial element defined by or secured to the base layer; securing the base layer to the skull to mount the fiducial marker on the skull such that the base layer conforms to the body surface; thereafter MRI scanning the patient with the fiducial marker assembly on the skull to generate corresponding image data; and thereafter identifying a physical location on the skull using the image data.

The application provides a method for identifying a physical location in the brain of a patient residing in physical space, the method comprising: providing a fiducial marker assembly residing in physical space and including: an/mri-compatible base layer that is mountable on the skull; and at least one MRI-visible fiducial element defined by or secured to the base layer, the fiducial marker assembly on the skull such that the base layer conforms to the skull's surface; MRI scanning the patient with the fiducial marker assembly on the skull to generate corresponding image data; and identifying a physical location in the brain using the image data, including: generating an image of the patient's brain in a logical space; determining in the logical space a desired entry location in the brain for insertion of instrumentation into the patient for planning surgical resection; and programmatically determining a physical location on the fiducial marker assembly corresponding to the desired entry location.

The application provides a computer program product for identifying a physical location in the brain of a patient using a fiducial marker assembly mounted on the skull surface and including at least one MRI-visible fiducial element, the computer program product comprising: a computer readable medium having computer readable program code embodied therein, the computer usable program code comprising: computer readable program code configured to generate an image of the patient's brain and the fiducial markers in a logical space, the image corresponding to an MRI scan of the patient with the fiducial markers on the skull surface; computer readable program code configured to determine in the logical space a desired trajectory line for insertion of instrumentation into the patient in order to plan surgical resection; and computer readable program code configured to programmatically determine a location of intersection between the desired trajectory line and the fiducial markers.

The application provides a system for designating a physical location in the brain of a patient, the system comprising: a fiducial marker including: an MRI-compatible base layer that is mountable on the skull surface; and at least one MRI-visible fiducial element defined by or secured to the base layer; and a controller adapted to communicate with an MRI scanner that is operable to scan the patient with the fiducial markers on the skull surface and to generate corresponding image data, wherein the controller is operable to process the image data from the MRI scanner to programmatically identify a physical location in the brain using the fiducial markers as correlated to a physical location in said brain.

The application provides a method for identifying a physical location in the brain of a patient residing in physical space, the method comprising: providing a fiducial markers residing in physical space and including: an MRI-compatible base layer that is mountable on a skull surface; and at least one MRI-visible fiducial element defined by or secured to the flexible substrate; mounting the fiducial marker on the skull surface such that the base layer conforms to the brain surface; MRI scanning the patient with the fiducial markers on the brain surface to generate corresponding image data; generating an image of said brain in a logical space; and programmatically determining an orientation of the fiducial markers in the logical space using the image data.

### BRIEF DESCRIPTION OF THE DRAWINGS

The foregoing and other features and aspects of the invention will be best understood with reference to the following description of a specific embodiment of the invention, when read in conjunction with the accompanying drawings, wherein:

FIG. 1 is a top perspective view of an exemplary fiducial marker assembly according to embodiments of the present invention.

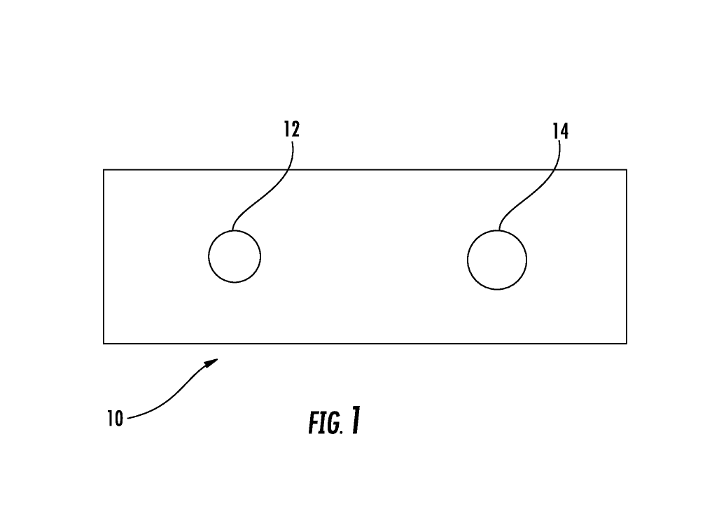

FIG. 2 is an enlarged, fragmentary, cross-sectional view of the fiducial marker assembly of FIG. 1.

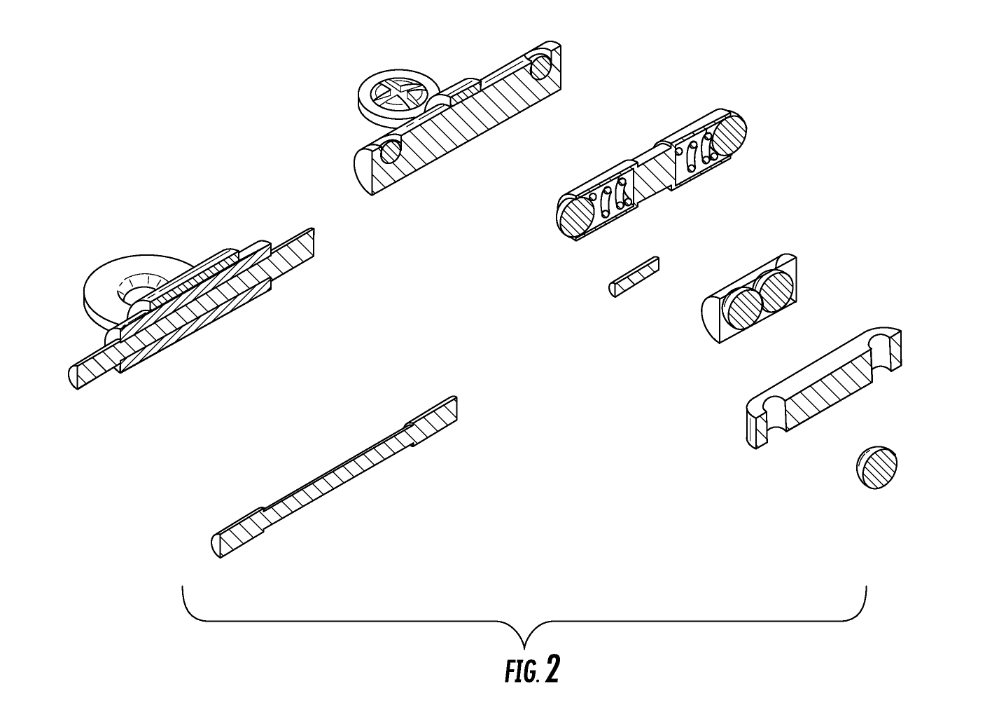

FIG. 3 is a view of the fiducial marker assembly depicted in FIGS. 1 and 2, according to further embodiments of the present invention.

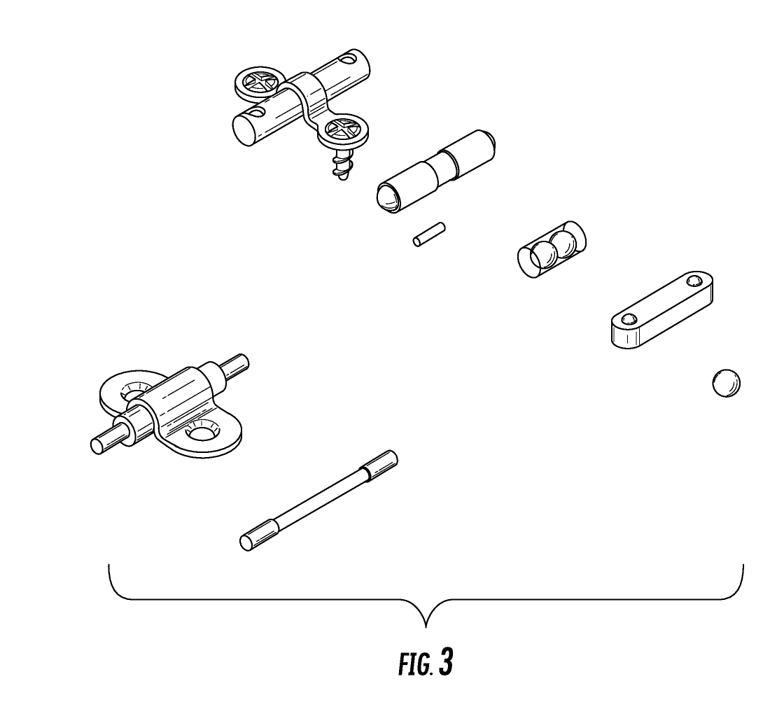

### DETAILED DESCRIPTION OF EMBODIMENTS OF THE INVENTION

The present invention now is described more fully hereinafter with reference to the accompanying drawings, in which some embodiments of the invention are shown. This invention may, however, be embodied in many different forms and should not be construed as limited to the embodiments set forth herein; rather, these embodiments are provided so that this disclosure will be thorough and complete, and will fully convey the scope of the invention to those skilled in the art.

Like numbers refer to like elements throughout. In the figures, the thickness of certain lines, layers, components, elements or features may be exaggerated for clarity.

The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the specification and relevant art and should not be interpreted in an idealized or overly formal sense unless expressly so defined herein. Well-known functions or constructions may not be described in detail for brevity and/or clarity.

It will be understood that when an element is referred to as being “on”, “attached” to, “connected” to, “coupled” with, “contacting”, etc., another element, it can be directly on, attached to, connected to, coupled with or contacting the other element or intervening elements may also be present. In contrast, when an element is referred to as being, for example, “directly on”, “directly attached” to, “directly connected” to, “directly coupled” with or “directly contacting” another element, there are no intervening elements present. It will also be appreciated by those of skill in the art that references to a structure or feature that is disposed “adjacent” another feature may have portions that overlap or underlie the adjacent feature.

Spatially relative terms, such as “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is inverted, elements described as “under” or “beneath” other elements or features would then be oriented “over” the other elements or features. Thus, the exemplary term “under” can encompass both an orientation of “over” and “under”. The device may be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

Exemplary embodiments are described below with reference to block diagrams and/or flowchart illustrations of methods, apparatus (systems and/or devices) and/or computer program products. It is understood that a block of the block diagrams and/or flowchart illustrations, and combinations of blocks in the block diagrams and/or flowchart illustrations, can be implemented by computer program instructions. These computer program instructions may be provided to a processor of a general purpose computer, special purpose computer, and/or other programmable data processing apparatus to produce a machine, such that the instructions, which execute via the processor of the computer and/or other programmable data processing apparatus, create means (functionality) and/or structure for implementing the functions/acts specified in the block diagrams and/or flowchart block or blocks.

These computer program instructions may also be stored in a computer-readable memory that can direct a computer or other programmable data processing apparatus to function in a particular manner, such that the instructions stored in the computer-readable memory produce an article of manufacture including instructions which implement the functions/acts specified in the block diagrams and/or flowchart block or blocks.

The computer program instructions may also be loaded onto a computer or other programmable data processing apparatus to cause a series of operational steps to be performed on the computer or other programmable apparatus to produce a computer-implemented process such that the instructions which execute on the computer or other programmable apparatus provide steps for implementing the functions/acts specified in the block diagrams and/or flowchart block or blocks.

Accordingly, exemplary embodiments may be implemented in hardware and/or in software (including firmware, resident software, micro-code, etc.).

Furthermore, exemplary embodiments may take the form of a computer program product on a computer-usable or computer-readable storage medium having computer-usable or computer-readable program code embodied in the medium for use by or in connection with an instruction execution system. In the context of this document, a computer-usable or computer-readable medium may be any medium that can contain, store, communicate, propagate, or transport the program for use by or in connection with the instruction execution system, apparatus, or device.

The computer-usable or computer-readable medium may be, for example but not limited to, an electronic, magnetic, optical, electromagnetic, infrared, or semiconductor system, apparatus, device, or propagation medium. More specific examples (a non-exhaustive list) of the computer-readable medium would include the following: an electrical connection having one or more wires, a portable computer diskette, a random access memory (RAM), a read-only memory (ROM), an erasable programmable read-only memory (EPROM or Flash memory), an optical fiber, and a portable compact disc read-only memory (CD-ROM). Note that the computer-usable or computer-readable medium could even be paper or another suitable medium upon which the program is printed, as the program can be electronically captured, via, for instance, optical scanning of the paper or other medium, then compiled, interpreted, or otherwise processed in a suitable manner, if necessary, and then stored in a computer memory.

Computer program code for carrying out operations of data processing systems discussed herein may be written in a high-level programming language, such as Java, AJAX (Asynchronous JavaScript), C, and/or C++, for development convenience. In addition, computer program code for carrying out operations of exemplary embodiments may also be written in other programming languages, such as, but not limited to, interpreted languages. Some modules or routines may be written in assembly language or even micro-code to enhance performance and/or memory usage. However, embodiments are not limited to a particular programming language.

It will be further appreciated that the functionality of any or all of the program modules may also be implemented using discrete hardware components, one or more application specific integrated circuits (ASICs), or a programmed digital signal processor or microcontroller.

The flowcharts and block diagrams of certain of the figures herein illustrate exemplary architecture, functionality, and operation of possible implementations of embodiments of the present invention. In this regard, each block in the flow charts or block diagrams represents a module, segment, or portion of code, which comprises one or more executable instructions for implementing the specified logical function(s). It should also be noted that in some alternative implementations, the functions noted in the blocks may occur out of the order noted in the figures. For example, two blocks shown in succession may in fact be executed substantially.

The term “MRI-visible” means that a device or feature thereof is visible, directly or indirectly, in an MRI image. The visibility may be indicated by the increased SNR of the MRI signal proximate to the device (the device can act as an MRI receive antenna to collect signal from local tissue) and/or that the device actually generates MRI signal itself, such as via suitable hydro-based coatings and/or fluid (typically aqueous solutions) filled cavities.

The term “MRI-compatible” means that a device is safe for use in an MRI environment and/or can operate as intended in an MRI environment, and, as such, if residing within the high-field strength region of the magnetic field, is typically made of a non-ferromagnetic MRI-compatible material(s) suitable to reside and/or operate in a high magnetic field environment.

The term “programmatically” refers to operations directed and/or primarily carried out electronically by computer program modules, code and instructions.

The term “fiducial marker” refers to a marker that can be identified visually and/or using electronic image recognition, electronic interrogation of MRI image data, or three-dimensional electrical signals to define a position and/or find the feature or component in 3-D space.

Fiducial markers in accordance with embodiments of the present invention can be configured to identify or designate a location on a body. The location may be identified in order to determine a desired position, orientation or operation of a guide apparatus. The guide apparatus may be used to guide and/or place diagnostic or interventional devices and/or therapies to any desired internal region of the body or object using MRI and/or in an MRI scanner or MRI interventional suite. The object can be any object, and may be particularly suitable for animal and/or human subjects.

In some embodiments, the guide apparatus is used to place implantable DBS leads for brain stimulation, typically deep brain stimulation hi some embodiments, the guide apparatus can be configured to deliver tools or therapies that stimulate a desired region of the sympathetic nerve chain. Other uses inside or outside the brain include stem cell placement, gene therapy or drug delivery for treating physiological conditions. Some embodiments can be used to treat tumors. Some embodiments can be used for RF ablation, laser ablation, cryogenic ablation, etc. In some embodiments, the interventional tools can be configured to facilitate high resolution imaging via intrabody imaging coils (receive antennas), and/or the interventional tools can be configured to stimulate local tissue, which can facilitate confirmation of proper location by generating a physiologic feedback (observed physical reaction or via fMRI).

Generally stated, some embodiments of the invention are directed to MRI interventional procedures including locally placing interventional tools or therapies in vivo to site-specific regions using an MRI system. The interventional tools can be used to define an MRI-guided trajectory or access path to an in vivo treatment site.

In some embodiments, MRI can be used to visualize (and/or locate) a therapeutic region of interest inside the brain or other body locations, to visualize an MRI-visible fiducial marker according to embodiments of the present invention, and to visualize (and/or locate) an interventional tool or tools that will be used to deliver therapy and/or to place a chronically implanted device that will deliver one or more therapies. Then, using the three-dimensional data produced by the MRI system regarding the location of the therapeutic region of interest and the location of the interventional tool, the system and/or physician can make positional adjustments to the interventional tool so as to align the trajectory of the interventional tool, so that when inserted into the body, the interventional tool will intersect with the therapeutic region of interest.

With the interventional tool now aligned with the therapeutic region of interest, an interventional probe can be advanced, such as through an open lumen inside of the interventional tool, so that the interventional probe follows the trajectory of the interventional tool and proceeds to the therapeutic region of interest.

The application provides an MRI-compatible fiducial marker assembly for identifying a location in a brain comprising: a MRI-compatible base layer that is mountable on the skull of a patient's body, one or more MRI-compatible fiducial elements attached to the base layer, wherein and at least one of said MRI-compatible fiducial elements are MRI-visible fiducial markers.

The application provides the above MRI-compatible fiducial marker assembly, including one or more screws to releasably attach the MRI-compatible fiducial markers to the skull.

The application provides the above MRI-compatible fiducial marker assembly, comprising two MRI-compatible fiducial markers that are MRI-compatible and MRI-visible fiducial markers.

The application provides the above MRI-compatible fiducial marker assembly, comprising two or more MRI-compatible screws to releasably attach the MRI-compatible fiducial marker assembly to the skull.

The application provides the above MRI-compatible fiducial marker assembly, wherein the base layer is made of a non-ferromagnetic material.

The application provides the above MRI-compatible fiducial marker assembly, wherein the one or more fiducial elements are made of a non-ferromagnetic material.

The application provides the above MRI-compatible fiducial marker assembly, wherein the non-ferromagenetic material is a metal.

The application provides the above MRI-compatible fiducial marker assembly, wherein the one or more fiducial elements are made of a non-ferromagnetic metal.

The application provides the above MRI-compatible fiducial marker assembly, wherein the one or more fiducial elements are made of gold.

The application provides the above MRI-compatible fiducial marker assembly, wherein the base layer is made of gold.

The application provides an MRI-compatible fiducial marker assembly for identifying a location, the fiducial marker assembly comprising: a base layer that is mountable on a patient's skull, the base layer having opposed upper and lower primary surfaces; and at least one MRI-visible fiducial element defined by or secured to the base layer.

The application provides the above MRI-compatible fiducial marker assembly, further including screws to attach the base layer to the skull.

The application provides the above MRI-compatible fiducial marker assembly, further including openings through the base layer through which screws can attach the base layer to the skull.

The application provides the above MRI-compatible fiducial marker assembly, wherein the screws may be used to release the base layer from the skull.

The application provides the above MRI-compatible fiducial marker assembly, further including indicia on the base layer corresponding to the at least one MRI-visible fiducial element.

The application provides the above MRI-compatible fiducial marker assembly, further including second indicia on the base layer corresponding to the at least one MRI-visible fiducial element.

The application provides the above MRI-compatible fiducial marker assembly, wherein the at least one MRI-visible fiducial element includes a plurality of MRI-visible fiducial elements defined by or secured to the base layer.

The application provides the above MRI-compatible fiducial marker assembly, wherein the MRI-visible fiducial elements are arranged in a defined pattern.

The application provides the above MRI-compatible fiducial marker assembly, further including at least one MRI-visible reference indicator to indicate an orientation of the assembly.

The application provides the above MRI-compatible fiducial marker assembly, wherein the MRI-visible fiducial elements are selectively discretely removable from the base layer.

The application provides the above MRI-compatible fiducial marker assembly, wherein at least one of the MRI-visible fiducial elements has a width and a height greater than its width to define a heightwise axis.

The application provides the above MRI-compatible fiducial marker assembly, wherein the base layer has a thickness in the range of from about approximately 1 mm to approximately 1 cm.

The application provides a method for identifying a physical location in the brain of a patient, the method comprising: providing a fiducial marker assembly including: a base layer that is mountable on a patient's skull, the base layer having opposed upper and lower primary surfaces; and at least one MRI-visible fiducial element defined by or secured to the base layer; securing the base layer to the skull to mount the fiducial marker on the skull such that the base layer conforms to the body surface; thereafter MRI scanning the patient with the fiducial marker assembly on the skull to generate corresponding image data; and thereafter identifying a physical location on the skull using the image data.

The application provides the above method, wherein the at least one MRI-visible fiducial element includes a plurality of MRI-visible fiducial elements defined by or secured to the base layer.

The application provides the above method, wherein the MRI-visible fiducial elements are arranged in a defined pattern.

The application provides a method for identifying a physical location in the brain of a patient residing in physical space, the method comprising: providing a fiducial marker assembly residing in physical space and including: an/mri-compatible base layer that is mountable on the skull; and at least one MRI-visible fiducial element defined by or secured to the base layer, the fiducial marker assembly on the skull such that the base layer conforms to the skull's surface; MRI scanning the patient with the fiducial marker assembly on the skull to generate corresponding image data; and identifying a physical location in the brain using the image data, including: generating an image of the patient's brain in a logical space; determining in the logical space a desired entry location in the brain for insertion of instrumentation into the patient for planning surgical resection; and programmatically determining a physical location on the fiducial marker assembly corresponding to the desired entry location.

The application provides the above method, further determining in the logical space the desired entry location includes determining a desired trajectory line; and determining the physical location on the fiducial marker assembly corresponding to the desired entry location includes determining a location of intersection between the desired trajectory line and the fiducial markers.

The application provides the above method, further including programmatically determining in the logical space the desired entry location and the desired trajectory line.

The application provides the above method, further including displaying the desired entry location and the desired trajectory line on a display device to an operator.

The application provides the above method, wherein the at least one MRI-visible fiducial element includes a plurality of MRI-visible fiducial elements defined by or secured to base layer.

The application provides the above method, wherein the MRI-visible fiducial elements are arranged in a defined pattern.

The application provides the above method, further including displaying the image of the patient's brain and a graphical overlay on a display to an operator, wherein the graphical overlay indicates at least a portion of the defined pattern of the MRI-visible fiducial elements.

The application provides the above method, further comprising forming a burr hole in the patient's skull relative to the portion of the brain intended for resection, as determined spacially in relation the fiducial markers.

The application provides the above method, wherein the mounting step comprises releasably attaching the fiducial marker assembly to the skull surface prior to the step of MRI scanning the patient with the fiducial markers on the skull.

The application provides the above method, further comprising MRI scanning the patient with the fiducial markers on the skull includes MRI scanning an MRI-visible reference indicator on the fiducial marker assembly to generate corresponding reference image data; and the method further includes programmatically determining an orientation of the fiducial marker using the reference image data.

The application provides a computer program product for identifying a physical location in the brain of a patient using a fiducial marker assembly mounted on the skull surface and including at least one MRI-visible fiducial element, the computer program product comprising: a computer readable medium having computer readable program code embodied therein, the computer usable program code comprising: computer readable program code configured to generate an image of the patient's brain and the fiducial markers in a logical space, the image corresponding to an MRI scan of the patient with the fiducial markers on the skull surface; computer readable program code configured to determine in the logical space a desired trajectory line for insertion of instrumentation into the patient in order to plan surgical resection; and computer readable program code configured to programmatically determine a location of intersection between the desired trajectory line and the fiducial markers.

The application provides a system for designating a physical location in the brain of a patient, the system comprising: a fiducial marker including: an MRI-compatible base layer that is mountable on the skull surface; and at least one MRI-visible fiducial element defined by or secured to the base layer; and a controller adapted to communicate with an MRI scanner that is operable to scan the patient with the fiducial markers on the skull surface and to generate corresponding image data, wherein the controller is operable to process the image data from the MRI scanner to programmatically identify a physical location in the brain using the fiducial markers as correlated to a physical location in said brain.

The application provides a method for identifying a physical location in the brain of a patient residing in physical space, the method comprising: providing a fiducial markers residing in physical space and including: an MRI-compatible base layer that is mountable on a skull surface; and at least one MRI-visible fiducial element defined by or secured to the flexible substrate; mounting the fiducial marker on the skull surface such that the base layer conforms to the brain surface; MRI scanning the patient with the fiducial markers on the brain surface to generate corresponding image data; generating an image of said brain in a logical space; and programmatically determining an orientation of the fiducial markers in the logical space using the image data.

The application provides the above method wherein the fiducial markers include MRI-visible reference indicators and programmatically determining the orientation of the fiducial markers in the logical space using the image data includes programmatically determining the orientation of the fiducial markers in the logical space using image data corresponding to the MRI-visible reference indicators.

That which is claimed is:

1. An MRI-compatible fiducial marker assembly for identifying a location in a brain comprising: a MRI-compatible base layer that is mountable on the skull of a patient's body, one or more MRI-compatible fiducial elements attached to the base layer, wherein and at least one of said MRI-compatible fiducial elements are MRI-visible.

2. The MRI-compatible fiducial marker assembly of claim 1, including one or more screws to releasably attach the MRI-compatible fiducial marker assembly to the skull.

3. The MRI-compatible fiducial marker assembly of claim 2, comprising one or more MRI-compatible fiducial elements that are MRI-visible.

4. The MRI-compatible fiducial marker assembly of claim 3, comprising two or more MRI-compatible screws to releasably attach the MRI-compatible fiducial marker to the skull.

5. The MRI-compatible fiducial marker assembly of claim 4, wherein the base layer is made of a non-ferromagnetic material.

6. The MRI-compatible fiducial marker assembly of claim 5, wherein the one or more fiducial elements are made of a non-ferromagnetic material.

7. The MRI-compatible fiducial marker assembly of claim 6, wherein the non-ferromagenetic material is a metal.

8. The MRI-compatible fiducial marker assembly of claim 7, wherein the one or more fiducial elements are made of a non-ferromagnetic metal.

9. The MRI-compatible fiducial marker assembly of claim 8, wherein the one or more fiducial elements are made of gold.

10. The MRI-compatible fiducial marker assembly of claim 9, wherein the base layer is made of gold.

11. An MRI-compatible fiducial marker assembly for identifying a location, the fiducial marker assembly comprising: a base layer that is mountable on a patient's skull, the base layer having opposed upper and lower primary surfaces; and at least one MRI-visible fiducial element defined by or secured to the base layer.

12. The fiducial marker assembly of claim 11 including screws to attach the base layer to the skull of a patient.

13. The fiducial marker assembly of claim 12 including openings through the base layer through which said screws can attach the base layer to the skull.

14. The fiducial marker assembly of claim 13, wherein the screws may be used to release the base layer from the skull.

15. The fiducial marker assembly of claim 14 including indicia on the base layer corresponding to the at least one MRI-visible fiducial element.

16. The fiducial marker assembly of claim 15 including second indicia on the base layer corresponding to the at least one MRI-visible fiducial element.

17. The fiducial marker of claim 16 wherein the at least one MRI-visible fiducial element includes a plurality of MRI-visible fiducial elements defined by or secured to the base layer.

18. The fiducial marker assembly of claim 17 wherein the MRI-visible fiducial elements are arranged in a defined pattern.

19. The fiducial marker assembly of claim 18 including at least one MRI-visible reference indicator to indicate an orientation of the assembly.

20. The fiducial marker of claim 19 wherein the MRI-visible fiducial elements are selectively discretely removable from the base layer.
